(19) United States
(12) Patent Application Publication
(10) Pub. No.: US 2011/0143393 A1
(43) Pub. Date: Jun. 16, 2011

(54) AUTOMATIC DEVICE FOR CARRYING OUT DETECTION REACTIONS, AND METHOD FOR DOSING REAGENTS ONTO MICROSCOPE SLIDES

(76) Inventors: Hartmut Merz, Gross Gronau (DE); Kay Van Der Wolk, Lubeck (DE)

(21) Appl. No.: 12/937,808

(22) PCT Filed: Apr. 14, 2009
(86) PCT No.: PCT/EP09/02729

§ 371 Date: Mar. 1, 2011

(30) Foreign Application Priority Data
Apr. 14, 2008 (DE) 10 2008 018 982.0

(51) Int. Cl. G01N 1/28; C12M 1/00

(52) U.S. Cl. 435/40.5; 435/283.1

(57) ABSTRACT

The invention relates to an automatic device to meter reagents onto a multitude of slides comprising holding devices for a multitude of slides which contain each a multitude of separated slots which are adapted to absorb cell or tissue samples; a dosing head unit with a multitude of reservoirs which are adapted to absorb liquid reagents and which have a metering valve at the end/side facing the slots to dose and apply the reagents into the slots; a positioning device to position the reservoirs of the dosing head unit above the respective slots of the slides.

### AREA OF THE INVENTION

The present invention relates to an automatic device for dosing reagents onto slides, whereby the slides contain a number of separate slots/indentations which are able to absorb cell or tissue samples. The device contains a positioned dosing unit to apply adequate liquid reagents in the slots of the slides. The invention furthermore relates to a detachable dosing unit which can be inserted into the device and a method for an automatic dosage of reagents in the separated slots of slides.

### PRIOR ART

Due to improved immunohistochemical and molecular biological analysis techniques in the recent past there is a higher demand of tissue examinations. Especially in the field of oncology diagnosis and treatment of cancer the focus is on gene-expression-profiling.

During this process tissue samples are analysed directly or indirectly or via tumour specific chips to see if specific genes are active or not or if genetic modifications (e.g. mutations) exist. Furthermore, the tissue and cell samples can be treated with antibodies or alternatively with DNA or RNA probes which then lead to specific proofs of the genes, gene-products (mRNA or antigens) via staining techniques. An immunohistochemical standard staining method is for example the so-called Avidin-Biotin-Complex (ABC) Method, which is described in Hsu, S. M. et al., J. Histochem. Cytochem, 29, 577-580. In the first step this method binds primary-antigen to the respective tissue-antigen. After one or several washing steps a biotinylated secondary-antigen is bound to the primary-antigen. After one or several further washing steps a preformed Avidin-Biotin-Enzyme-Complex is superimposed which adsorbs the biotin-molecule of the secondary-antigen with high-affinity. Again, one or several washing steps follow. After that a Chromogen is superimposed which shows a stain reaction with the Avidin-Biotin-Enzyme-Complex by creating a visible stained reaction product. Molecular biological procedures comprise the In-situ-Hybridisation (ISH) and the fluorescence-in-situ-Hybridisation (FISH), at which nucleic acids ergo RNA or DNA, e.g. are verified in tissues or individual cells. Thereto synthetically made probes of nucleic acids are used which hybridize via base pairs and nucleic acids to be verified. At the FISH-method the probe is verified by means of a fluorescent dye.

In performing gene expression analysis, one can exploit the fact that a thousand or even all genes of a total of 25,000 to 30,000 genes of a cell can be contemporaneously analyzed. It is assumed that only half of the approximately 25,000 genes of a cell are active and thereof only a few hundreds are decisive for diseases or tumors. For instance, with about 200 genes one can significantly delineate mantel cell lymphoma from all other known lymphomas (about 40 different entities are know, which can be further divided in sub classes). With a similar amount of different genes, mantel call lymphomas can be distinguished from other tumors. Moreover, with some hundred genes can one can differentiate between reactive lymphocytes and neoplastic lymphocytes of a mantel cell lymphoma.

Based on statistical evaluation of the results of said global expression analyses the amount of data can be significantly reduced. Moreover, genetic fingerprints of e.g. tumors or etiopathology are created which can be used as markers in diagnostic medicine. In doing so, the validity of the analyses is generally not significantly reduced. For instance, from about 30 genes known to be relevant for highly malignant b-cell lymphomas one can select three genes, which correlate greatly with a positive and negative prognosis respectively. A mathematical formula for prognosis based on those six genes connected with either a positive or negative factor, may thus predict the patient's chance of survival with a high degree of certainty. Furthermore, this prediction can be integrated in the clinical and therapeutic assessment and be thus of additional benefit for the patient.

As tissues and cells normally die within a short period of time when extracted from the organism and thus underlie the process of necrosis damaging organelles, cell structures and tissue structures, tissue analyses are mainly performed on so called FFPE-tissue (formalin fixed and paraffin embedded). Therefore, about 250 well-established antibodies are available for said FFPE-material. However, so called algorithms are frequently used for analysis. In doing so, tumors are first of all examined using conventional morphologic standard staining. The pathologist ends up with either a tentative diagnosis or with a plurality of differential diagnoses, for instance taking three of four different diagnoses into account. For a final diagnosis, a variable amount of immunohistochemical or molecular biological staining reactions are carried out. To be of certain economic efficiency, said staining reactions are carried out with so called algorithmic antibody-panels, i.e. expedient groups of antibodies are used. An algorithmic antibody-panel may comprise four to six antibodies. Different antibody-panels may be logically connected (e.g. hierarchically) or aggregated in different groups directed to specific diseases. In moving from one panel to the next, the pathologist elaborates the diagnostic problem in a sophisticated way until the final diagnosis is set.

Accordingly, 10 to 50 different examinations and analysis are carried out on the patient's tissue (e.g. within the frame of so called tumor specific diagnosis or prognosis panels). Due to the prevailing cost pressure in public health sector, said examination has to be performed in a time efficient and cost reducing way.

It has been proposed to use automatic staining devices, in order to accomplish such tissue analyses.

The U.S. Pat. No. 6,495,106 describes an automatic staining device which has a three-dimensional mobile arm which carries a head with a recess. This head unit comprises a reagents head with a tip in order to apply the reagents. Furthermore, the head unit comprises a washing-tip and a blowing-tip to accomplish the different steps of a typical proof reaction.

A similar device is described in the U.S. Pat. No. 5,948,359, whereby the reagents-head unit can be equipped with one-way pipette tips which are charged with a special holding-device.

These devices are less practical to carry out proof-reactions with a high number of slides which each have a multitude of reaction-zones to absorb the cell or tissue samples, because the entire analysis would take very long as there is only one reagent tip. Furthermore, from a diagnostic point of view there is need to perform a multitude of analyses of tissue samples of one single tissue for an individual patient in order to be able to draw and verify a diagnosis, according to the above described method. Nevertheless, for this purpose, the tissue samples of material originating from the same patient need to be mixed with different antibodies or probes, to be able to isolate and eventually diagnose the specific tumour. Concerning the devices described in the U.S. Pat. No. 6,495,106 and in the U.S. Pat. No. 5,948,359, a change of the pipette tip of the reagents tip is necessary if the proof-reagent is exchanged. This results in a higher consumption of the pipette-one-way tips, as well as in an increase of time and eventually of the costs for the entire analysis.

The US 2008/0,038,836 A reveals a staining device which is practical for a multitude of slides, comprising a multitude of process stations which are vertically located in a magazine. Additionally, the device contains a tray with a multitude of slides which are located horizontally on one level and being separated from each other. During processing, the tray with the slides can be moved between, into and out of the individual process stations by a transport system. The device contains, for example, one process stations or a plurality of process stations for drying and heating, one for dewaxing and deparaffining of FFPE-tissue, and one staining process stations or a plurality of staining process stations. The staining station shows a row of nozzles which are connected to containers containing staining-reagents or rinse-agents and which are used to apply those reagents or agents onto the slides. The device described in the US 2008/0,038,836 A is very complex and the transport of the tray holding the slides between the individual process stations is time consuming. Furthermore, only a limited number of, for example, nozzles exists which need to be changed if different reagents are used. The reagents are drained out of the reservoirs into the nozzles via hose lines, thus complicating the handling of the X-Y-Z-positioning system.

The US 2006/0,269,447 reveals a robot to apply liquids onto microtiter plates which can be used for example as ELISA plates (enzyme-linked immunosorbent assay). The plates have for example 96 or 384 micro-slots. The robot comprises a pipetting head which is fixed to a positioning system. At a park station, the pipetting head collects the tray with the pipette-tips and drives these by means of the positioning device to the microtiter plate where the reagents are dosed into the micro-slots of the plate. The pipette tips which have a volume of 50 to 250 μl can be dropped after the liquid dosing. The pipetting head moves to the park station and collects a new tray with pipette tips. Similar devices are described in the DE 100 40 849 and in the US 2004/0,097,010.

Such devices known from the prior art are not appropriate to conduct proof-reactions with a multitude of cell or tissue samples arranged on slides. The reloading of the tray with pipette tips after each individual dosing process above a single microtiter plate is highly time-consuming. During reloading the pipette tips are dropped and replaced by new ones. This leads to substantially increased processing costs.

Considering the aforementioned prior art, one objective of the present invention is to provide an automatic device to dose reagents onto a multitude of slides at which the above described disadvantages should be eliminated completely or at least to the greatest possible extent. Another object of the invention is to provide a device which is able to conduct the process of dosing reagents onto slides with a proof-reaction at a multitude of tissue samples on a number of slides in an effective and time-efficient way. This is as to handle the increased demand of immunohistochemical and molecular biological analyses of cells and tissues within the healthcare system and to preserve resources at the same time. Further objects of the invention are clear to the specialist from the following description.

### BRIEF DESCRIPTION OF THE INVENTION

The present invention relates to an automatic device for dosing reagents onto a multitude of slides, comprising:
holding devices for a multitude of slides which each contain a multitude of separated slots which are adapted to absorb cell or tissue samples,
a dosing head unit with a multitude of reservoirs which are adapted to absorb liquid reagents and a metering valve at the end/side facing the reaction zones to dose and apply the reagents into the slots, and
a positioning device to position the reservoirs of the dosing head unit over the respective reaction zone of the slides.

Furthermore, The present invention relates to a dosing head to be used in an automatic device to dose reagents onto a multitude of slides comprising a multitude of reservoirs which are adapted to absorb liquid reagents and a metering valve at one end/side to dose and apply the reagents onto the slides, and which have at the opposite end of the metering valves a gastight cover at which the middle capacity of the reservoir is at least 1.5 ml and preferably at least 2.5 ml.

The present intervention relates also to a method for dosing reagents onto a multitude of slides by the means of an automatic device according to the invention, in which
the slides are inserted into the device,
the reservoirs are filled with reagents,
the dosing head is positioned over the slots of the slides by means of the positioning device, and
the reagent is dosed into the slot at which the volume of the reagent is smaller as the capacity of the slot.

### DETAILED DESCRIPTION OF THE INVENTION

The present invention relates to an automatic device for dosing reagents onto a multitude of slides which have each a multitude of separated slots. The slots are adapted to absorb tissue or cell reactions at which then especially immunohistochemical or molecular biological proof reactions are conducted with added reagents.

Slots which are filled with tissue or cell samples are designated as reactions-zones. In the context of the present invention, the expression “multitude” relates to at least the number of three, preferably to at least the number of six and more preferably to at least the number of eighteen.

Slides which are appropriate to be used in the present invention embrace e.g. a preferably transparent level basis plate on which e.g. a uniform grid of bars is located to provide slots which are separated from each other. The slide should be made of a homogeneous transparent material as e.g. glass of polyethylene, a Teflon layer or a layer of another preferably hydrophobic polymer material in which the slots are imbedded. The slots can be inserted in an originally homogeneous thick layer e.g. by drilling or milling, or the slides can be produced integrally e.g. by injection moulding. The slide can also have an evenly thick basis layer on which bars are positioned in a grid in which then an adapted hydrophobic polymerizable precursor of a polymer is applied and subsequently polymerised e.g. by the screen printing method.

The slides have a number of slots e.g. 6, 18, 24, 72 or even more. The cross-sectional areas of the slot can be even as well as uneven. Nevertheless, even cross-sectional areas are preferable e.g. squares, rectangles or circles. The recess depths of the slot can vary in terms of the cross-sectional area if applicable, but mostly it is constant. Ideally all slots of a slide have the same form. Additionally, according to the invention standard slides can be used in the device as well.

The dimensions of the cross-sectional area and the recess depth of the slot are chosen in a way that the slots have a volume of preferably 5 to 150 μl.

The dimensions of a slot with square or rectangle cross-section can preferably be between 3 to 10 mm×3 to 10 mm, the depths can vary between 0.1 and 2.0 mm.

The slots on the slides are separated by bars which can have a regular or irregular form and which can have an even or uneven thickness. Preferably, the bars form a regular grid in which the bars stand vertically on each other, if the slot has a square or rectangle cross-section.

Preferably, the dimensions of the slides comply with standardized formats. The dimensions of the slides are 26 mm×75 mm. Preferably, a description or indication field is located at one end of the slide which can be labelled or tagged with an one- or two-dimensional code-label, a RFID-Chip or similar.

For example, a slide comprises 18 slots. The slots are arranged in a regular matrix of 3 columns and 6 rows. The slots are arranged equidistantly in a row. The distance between the individual rows is the same. A preferable design of a slide with 6 slots can be easily deducted from the afore-mentioned slide with 18 slots, if the three slots of one row are aggregated into one. In doing so, a stripe-design of 6 parallel striped slots is obtained. The slots on the slide are formed by bars and a basis plate at which the height of the bars corresponds to the recess depths of the slots. The height of the bars is greater than the height of the carrier foil and of the tissue and cell samples applied thereon. The relation of these dimensions is preferably at least 1.2, more preferably at least 1.3 even more preferably at least 1.4. The volume of the slot preferably exceeds the sum of the volumes of tissue and cell samples and the carrier foil segment by at least 1.5-times and preferably by at least a factor of 2.

To produce flat/extensive tissue sections FFPE tissue or cell material are primarily used, of which said flat/extensive tissue or cell sections, with a thickness of 0.5 to 5 μm and especially of 0.5 to 2.5 μm can be obtained by means of a microtome. It is preferred to mechanically reinforce the flat/extensive tissue sections by applying them to a preferably malleable carrier foil, e.g. a polycarbonate film. Subsequently cell or tissue segments can be punched out of the flat/extensive tissue section. In doing so, the horizontal expansion of the tissue segment vertical to its thickness is chosen in that in a subsequent step the tissue segments can be collected by a separation unit and can be placed into the slots. The tissue segments can be fixed in the slots for instance with a UV curable adhesive.

By using an appropriate hydrophobic grid the volumes of tissue and foil and adhesive can be almost identical (almost even surface), as the applied liquids would always remain pre-bent over the tissue in the reaction field by the hydrophobic forces due to surface tension. Hence, no contamination takes place. An almost leveled surface bears the advantage that at the final coverslipping process (permanent coverslipping of slides) a relatively small quantity of mounting medium needs to be used. The latter contains solvent which evaporates over time and which causes shrinking artefacts. Air is sucked into the slide, which hence can no longer be examined under a microscope. Air exhibits another refractive index as glass or mounting medium, whereas the mounting medium has a similar refractive index to glass.

As the formalin-fixed tissue or cell materials, embedded in the paraffin matrix, out of which the flat/extensive tissue sections are obtained are typically inhomogeneous, i.e. they exhibit unevenly formed sick or healthy areas in three dimensions, it is preferred to punch tissue or cell segments out of several flat tissue-sections of one and the same tissue or cell material and transfer this in the slot of the slides. Hence, it is ensured that a representative image/illustration of the tissue sample is examined. If needed the flat tissue or cell sections can visually be inspected by pathologist before segments are punched out. That way, flat sections which only contain paraffin and almost no other tissue or cell material or those sections which only contain healthy tissue cell material could be discarded.

The WO 2007/134,814 reveals for example an automatically working device for repeatable production of cell or tissue samples which are arranged on slides and to be analysed. As in particular FIGS. 1 and 2 are taken from the WO 2007/134,814, distinct reference is made to said application.

The device has holding devices to collect a multitude slides, i.e. from 1 to 200 slides. The holding devices can be fixed at one or several trays which can then be loaded with slides outside the device and reinserted into the device. It is also possible that the holding device and the trays are firmly connected to the holding device so that the slides are directly inserted into the holding device which is embedded in the device.

The size, form and design of the trays are variable. The trays are either one-piece trays or can consist of several pieces. One-piece trays, which are mainly rectangle trays, have a substantially rectangle frame which shows a plurality of parallel arranged strips or a cross-shaped grid on which a plurality of slides are fixed. If the size of such a one-piece tray corresponds to the size of the reaction area of the device, only one tray needs to be inserted into the device per analysis process which facilitates the handling of the device. Concerning multi-piece trays, the frames, individual strips or cross-shaped grids or grid parts are not firmly fixed but are detachable. This facilitates the loading of the trays as only one tray needs to be inserted into the device. In another embodiment two or more trays can be inserted. This is preferable, if for instance the slides are loaded with cell or tissue samples at different process stations.

The holding device connects the slides in a detachable way with one tray or a plurality of trays or with the device itself. In particular, mechanical holding devices are used. The slides can be fixed with clips or similar holding devices. Furthermore, mechanical fastening systems with female or male fastening elements can be used, with one fastening element fixed to the slide and the other one to the tray(s) or to the device. Accordingly, the slides can show circular through-holes which can engage the respective pins on the tray(s) or the device. The slides can also show e.g. on the bottom side opposite to the reaction zones mushroom-shaped micro-hooks which engage a fibrous fleece which is positioned on the tray(s) or on the device. With appropriate positioning or conducting elements it can be ensured that the slides are mainly fixed onto the device in the same alignment.

In consequence of the configuration of the holding devices, the configuration of the slides on the tray(s) or in the device can be regular or irregular whereas a regular configuration is preferred In a preferred embodiment, the configuration of the holding devices is chosen in that the slides form a regular rectangular matrix with rows and columns. Preferably, the slides are inserted in that the description fields which contain e.g. a code or a similar identification element always have the same direction.

Preferably, the device comprises a device for moving individual slides or groups of slides by vibration, whereby reagents dosed into the reaction zones can be mixed and additionally contribute to a constant wetting of the tissue and cell samples with reagents in the reaction zones. Furthermore, any rinsing fluid residues can be mechanically shaken off, if needed after a rotation of the revolving stored slides by e.g. 180°.

Preferably, the device additionally contains a device for heating individual slides or groups of slides. The heating can be conducted either in the simplest way by ambient air heating, or in a more complex way by for instance devices like an electric heating applied in proximity to the slides or by applying IR-radiation. A heating of the slides and therewith of the reaction zones is preferable, as this can reduce the necessary incubation time of the tissue or cell samples with the antibodies. Due to denaturing of antibodies or detachment of the hybrids above the melting temperature, the temperature is limited to 42° C. For the immunohistochemistry in general for the FISH analyses, an operating temperature of approximately 37-42° C. is preferable. Nevertheless, higher temperatures can be used for ISH-analyses. An acceleration of the reactions can also be obtained by a concentration increase of the reagents.

Immunohistochemichal antibody incubations typically need some 20 to 60 minutes (average 30 min), whereas molecular biological hybridisations typically need many hours or days. Therefore it is not practical to undertake immunohistochemical and molecular analyses at the same time. Additionally, there should be between 100 and 200 antibodies typically available for immunohistochemical analyses, whereas only 10 to 30 probes are typically needed for FISH and ISH analyses. Hence, different dosing heads are preferably used for immunohistochemical and molecular analyses. In view of these differences, it might be preferable to use devices according to the invention, which are optimised for the respective requirements for the immunohistochemical or molecular analyses. A device according to the invention with two sub-modules at which parts of the hardware and the software can be used in parallel is of preference.

Preferably, the holding devices are arranged such that the top face of the slides is mainly leveled with the slots/reaction zones. This bears the advantage that the positioning device which carries the dosing head only needs to facilitate two dimensional movements. The positioning device is primarily a conventional Cartesian X/Y-System which has e.g. a stepper motor. Typically, a positioning accuracy of ±0.25 mm is sufficient.

Additionally, according to the invention the device comprises a dosing head with a multitude of reservoirs which is fixed at the positioning device. Those reservoirs contain liquid reagents appropriate for reactions such as stain proof reactions.

Preferably, the dosing head has between 1 and 288 reservoirs, and more preferably with the number of reservoirs being a multiple of 6.

The reservoirs each have an end facing the slots of the underlying slide which each in turn having at least one metering valve with an outlet device to dose and insert the reagents into the slots or reaction zones. Preferably, the end of the reservoirs facing the metering valve the reservoirs have a gastight catch or top cover. The reservoirs can either have an individual gastight catch, or several to preferably all reservoirs have a single shared gastight catch. Preferably, the catch or catches have connexions with induction valves at which a positive gas pressure can be generated via one or several conductions. In doing so, the reagents in the reservoirs are primarily impinged with a positive gas pressure so that the liquid reagents can be dispensed with constant speed into the slots or reaction zones when the metering valves at the other end of the reservoirs are opened. Either air or an inert gas, for instance N₂, can be used The desired quantity of the reagent to be dosed can be determined by the opening time of the metering valve when it is adequately calibrated.

In a preferred embodiment, the dosing unit contains a dosing control.

It is recommended to verify, whether the measured off reagent has been effectively dispensed, at a position at or below the outlet of the metering valves, particularly using optical sensors if. The measuring off itself is controlled by obstruction of a light beam, especially infrared light beams, to see whether the dosing process has been successfully started and terminated. In order to obtain a reliable and for example colour-independent output signal, the increase (volume over time (dV/dt)) is analysed via e.g. an ND-converter (Analog-Digital-Converter). Additionally, a time course analysis of the metering process is possible at which the period of light beam obstruction is measured. Based on the measured obstruction time and the predefined and controlled system pressure, the dispensed volume is calculated. This is advantageous in that the reliability of the device is increased and a feed-back of the respective filling level of the individual reservoirs can be facilitated.

Alternatively, it is also possible to envisage a capacitive flow sensing system at which a measurable capacity variation is detected and used for control instead of an optical flow control system.

Preferably, the measuring valves are arranged in a valve block at the lower end of the reservoirs. It is preferred that the measuring valves are not furnished with dosing tips, as a dead space volume would thus be generated (namely the one of the dosing tip). Hence, the metering is primarily conducted via the openings in a perforated plate which is positioned below the valves. If needed, the outlet opening of the metering valve can show micro- or dosing needles, whose diameters are significantly smaller than the cross section of the reaction zones or of the reservoir of the dosing unit in order to limit the dead space volume.

Furthermore, an optical bubble control can be provided, to detect undesired air bubbles in the eluate.

The reservoirs of the dosing unit show a cross-sectional area and thereto a mainly vertical height between the two ends, which comprise the metering valve or the gastight catch. The configuration of the reservoirs at the dosing unit is chosen preferably in adaptation to the respective design of the slides in that adjacent reservoirs can be positioned over at least two, preferably adjacent slots or reaction zones of the slides. Furthermore, the cross-sectional area of adjacent reservoirs in adaptation to the dimensions of the reaction zones and the bars lying in between is to be chosen in that the slots or reaction zones of adjacent reservoirs can simultaneously be impinged with reagents at which the reaction zones are ideally adjacent too. If necessary, a simultaneous impact of staggered reaction zones is possible too. In this case the configuration of the reservoirs in the dosing unit is to be chosen in that preferably at least three or four and more preferably at least six adjacent slots or reaction zones can simultaneously be impinged with reagents out of the reservoirs.

Devices according to the invention which facilitate such a multi-parallel metering of reagents into different slots or reaction zones of one or several slides arranged in the reaction room are especially preferred. With the multi-parallel operating mode, a shorter analysis time and hence a substantial decrease of the analysis costs can be obtained. Additionally, the multi-parallel applied reagents can primarily be aggregated in algorithmic panels by which a particularly effective and targeted pathologic diagnostics is facilitated. The device according to the invention is therefore better suitable for mass screenings in the health care system than the automatic pipetting-machines currently available in the prior art.

Nevertheless, the device according to the invention as well allows for handling standardized slides with only one sample without restriction and with substantially lower cost. Thereby, one valve is opened and dispensed above the sample material, hence, perfect flexibility is added to the afore-mentioned advantages. For example, it could be necessary that when a very heterogeneous and non-portionable tissue is involved, a section needs to be processed “in total” or it could be necessary to process external sections which cannot be further manipulated.

The volume of the reservoirs is determined by the cross-sectional area and its height, i.e. the distance between the end preferably covered by a gastight catch and the opposite end limited by a metering valve. The volume of the reservoirs is dimensioned as to permit preferably 1 to 200 individual reactions, like ABC-staining proof reaction cycles without refilling of the reservoir being necessary. This also presents another advantage over known devices from the prior art at which the pipette-tip typically needs to be abandoned after a single-dosing and new tips are to be loaded for the next pipetting process. According to the invention, the time consuming movement of the pipette-head to the reloading station after each dosing process and the discharging of the one-way pipette-tip, being undesired for cost reasons and environmental reasons, is not required any more. According to the invention, the device can be processed in a more time and cost efficient way as conventional pipetting-devices. The device according to the invention is therefore much more suitable for mass screenings in the health care system than the automatic pipetting-machines currently available in the prior art.

The reservoirs of the pipetting head are dimensioned in that all slides inserted into the device with at least one tray, where applicable can be impinged with proof-reagents, the latter taken from the reservoirs without an interim refill of the reservoirs being necessary. Supposing for example that the device can collect a tray with 96 (8×12) slidesand that each slide has 18 (3×6) reaction zones with a respective volume of about 10 to 20 μl and that per reaction zone—without taking into account the rinsing process—reagents need to be applied in up to six steps, this results in a total dosing-head volume, i.e. the sum of the volumes of all reservoirs, of approximately 100 to 200 ml. Assuming that approximately 120 reagents are needed in 120 reservoirs, this results in a volume of about 5 ml per reservoir supposing a regular consumption of all reagents. At the six-slot slides about 30 to 50 μl are used.

Independently of these considerations, the reservoirs are preferably constructed in that the medium volume is at least 1.5 ml, preferably at least 2.5 mi, more preferably at least 4 ml and even more preferably at least 5 ml. All reservoirs can have the same volume.

As individual reagents as e.g. the secondary-antibody reagent and e.g. the rinsing fluid which are mainly the same for all staining reactions, are needed more often and in larger quantitites as other reagents (such as the primary reagents at the ABC-method) these more frequently needed reagents can be multiply stored in the dosing head or in bigger containers outside the dosing head. For example, it is also possible to attach reservoirs which can contain larger volumes in or at the case of the device itself, hence the therein stored secondary-antibody or rinsing fluid does not need to be moved with the dosing head itself. In order to meter the required larger volumes of the proof-reagents (secondary or if applicable tertiary reagents) as well as of the substrate, the counter-staining reagents or of the rinsing fluid, these liquids can be conducted via several tubes. Said tubes are pulled over drag-chains through the Cartesian system to a position close to the dosing head. It is especially recommended to have adequate holdings for the outlets of the tubes at the dosing head itself so that these can be moved with the dosing head over the slides lying below. It is conceivable in turn to install a valve head, where applicable, with dosing control at the tube-outlet itself. This valve head permits the metering of the required volumes of proof reagents (secondary or tertiary reagents) as well as of the substrate, the counter-staining reagents or of the rinsing fluid. Alternatively, it is conceivable to attach the valve head at the reservoir itself, in particular to minimize the weight transported by the dosing head. Furthermore, it is preferable that rinsing of these tube systems is possible.

If the reservoirs do not have an identical volume, the smallest reservoir has a volume of preferably about 5 ml or more.

In a preferred embodiment, the dosing head can be constructed as a “sandwich” with exchangeable plates or blocks and can for example include a caulking cover, a block unit of reservoirs, a block of metering valves and/or a block of flow sensors. The individual plates or blocks can be exchanged if needed e.g. in case of worn valves or reservoirs permanently soiled by deposits. This permits a high maintainability and modularity concerning potential extensions. Additionally, those modular possibilities for exchange of the individual plates or blocks permit a cost-saving operation of the device according to the invention, especially when the reservoirs are soiled only the dosing heads but not the cost-intensive metering valves need to be exchanged.

The dosing head is preferably detachably mounted to a dosing head holding. This facilitates the exchange of e.g. empty dosing heads or the insertion of a dosing head with other reagents. The dosing head itself can preferably be marked and hence indentified via machine-readable codes.

If needed, the reservoirs can be equipped with a level sensor. Alternatively, the entire system can be equipped with a filling level detection device, e.g. a triangulation sensor. After removal of the upper catch of the reservoir of the dosing head (cover) the individual reservoirs of the dosing head can be moved underneath the filling level detection device and the filling level of the individual reservoirs can easily and effectively be determined.

In a preferred embodiment, partly or completely drained dosing heads which are fixed in a dosing head holder and the reservoirs fixed in the dosing head respectively, can be moved to a refill station by the positioning system. In order to avoid manual refilling of the relatively small volumes in the dosing head and a therewith associated error source (accidental inversion of reagents at a manual refill) the refill station is preferably automated and is supported by corresponding software. The dosing head fixed onto the Cartesian system is moved below the refill station which is equipped with larger reservoirs of the used reagents. The individual reservoirs in the dosing head are automatically and individually refilled. In doing so, the volumes previously drained out of the individual reservoirs are known to the control system of the device (remanently memorised), especially by assessing the outlet via the dosing control and/or via the level sensors in the individual reservoirs. Prior to the filling operation, only the upper catch of the reservoir (cover) would have to be removed which can basically also be done automatically.

The reservoirs of the dosing head are preferably numbered. In a preferred embodiment, the individual reservoirs each have identification elements and thus can be activated individually or in groups.

Immunohistochemical or molecular biological proof reactions are generally multi-step reactions at which each reaction step is followed by a rinsing step. The immunohistochemical standard staining reaction, the so-called ABC-method (Avidin-Biotin-Complex-Method), consists for example of at least 3 reaction steps (dosing with primary-antibodies, secondary-antibodies and tertiary-reagents) whereby each individual reaction step is followed by e.g. 3 rinsing steps and after each rinsing step is preferably followed by a light drying step (no complete desiccation). The molecular biological standard procedures ISH (In-situ Hybridisation) and FISH (Fluorescence In-situ Hybridisation) respectively, are also multi-step procedures.

In a preferred embodiment of the device of the present invention, a rinsing tank filled with rinsing fluid intended to conduct the rinsing steps, is located below the slide level. In this embodiment the slides arranged in trays, where applicable, are transferable individually and/or together with other slides from a first position adapted to conduct the proof reactions to a second position located in the container.

Preferably, the slides are matrix-like arranged in rows and columns, whereby the strips with the slides which form the columns can individually be lowered and can therefore be brought into contact with the rinsing fluid. Of course, other parts of the slide-matrix such as e.g. rows, parts of columns, etc. can be arranged to be lowered. The slide-strips which form the columns or rows can alternatively or additionally be pivotally positioned, hence the slides can be transferred into the rinsing fluid by rotation. For temporal reasons, it is preferable that individual slides or groups of slides can be lowered as it is hence possible for the dosing head to activate non-lowered slides and impinge them with reagents. Additionally, for a specific reaction step the application time of the reagents onto the tissue or cell samples in the individual reaction zones in principal should be the same.

The rinsing tank positioned below the slide-level can be made of e.g. stainless steel (VA-steel). Preferably, it comprises an in- and outlet for the rinsing fluid in order to avoid a contamination of the reaction zones by contaminated rinsing solution. The solution can mainly consist of water to which detergents and e.g. NaAcid can be added to avoid microbial contamination. The continuous exchange of the rinsing solution also prevents microbial contamination.

Preferably, a a constant liquid level of the rinsing fluid in the rinsing tank is adjusted by means of a level sensor. The level is adjustable and can be adapted to the position of the slides turned by 180° and of the reaction zones pointing to the rinsing tank.

The inventive rinsing mechanism facilitates and accelerates the rinsing process by transferring one or several slides in the rinsing tank with a large surplus of rinsing fluid. A contamination which might be caused by diverting the individual reagents from one slide to another according to the rinsing process, was not observed, if the volume of the rinsing tank is sufficiently large. The volume of the rinsing tank is preferably 100 times as large as the volume of the rinsed reaction zones. Assuming that 10 slides each with 18 reaction zones, each with a volume of 50 μl are rinsed simultaneously, the volume of the rinsing tank should at least be 1000 l. Independently of this estimation, the rinsing tank preferably has a volume of at least 500 ml, more preferably of at least 1 l and even more preferably of at least 1.5 l.

After each rinsing process, the reaction zones typically contain rinsing fluid residues which should preferably be removed to avoid an unwanted dilution of the reagent in the following reaction step or even an overflow of the reaction zones. The latter could lead to a contamination of other reaction zones of the slide. To remove the rinsing fluid, a current/stream with air or preferably an inert rinsing gas like N₂ can preferably be generated above the slides. The impingement with air or with a rinsing gas is preferably conducted in that a laminar air or gas flow is generated. Nevertheless, it is important that the reaction zones never completely desiccate which could lead to denaturation of the cell or tissue samples.

To avoid desiccation of the cell or tissue samples the reaction area contains a cover to seal the entire reaction area of the device. The cover is preferably used in combination with the rinsing tank positioned below the level of the slides. By slight heating of the rinsing fluid in the rinsing tank, the fluid partly evaporates and reaches the air or gas area above the level of the slides. This process creates a tempered and humid reaction chamber.

Preferably, the device comprises a central control panel like a ¼ or 1/1 VGA-Touch-Panel. A software interface with the superordinate pathology software is not compulsory as the device conducts the analyses in an autarkic way without a data transfer out of existing systems being necessary.

Nevertheless, the following interfaces are preferably provided:
An USB/Ethernet-interface for a potential logging and interaction with a superordinate software (Pathology software or e.g, Patho-Link and/or dgl.), as for instance requirements/orders of an expert could be directly fed into the system at the microscope to be further processed by the machine in the laboratory according to the encoding and without the risk of being mix-up. In this way logistical advantages can be obtained. The Ethernet-interface can be configured as a TCP/IP-interface in order to connect the device according to the invention to peripherals as for example a printer or other data technical units.
An ISDN/Analogue/Ethernet—interface for remote maintenance.

Particularly, besides the standard known bidirectional cable based connections also modern bidirectional wireless connections especially per radio contact generated data connections are proposed.

The FFPE-microtome-cuts need to be pretreated prior to an immunohistochemical or molecular biological analysis. Firstly, the sections are dewaxed by means of a solvent (e.g. 3×10 min. Xylol bath), then they are transferred into an aqueous solution over a descending alcoholic sequence (e.g. 100%-96%-70%-70% Ethanol for 1 min respectively) and eventually they are treated in a heat mode in a buffer solution (microwave mode, steamer or steam cooker can be used therefore).

The pretreatment of the sections or of the tissue or cell samples dropped into the slots can be done in the device according to the invention. The necessary reagents are to be filled into the reservoirs of the dosing head and the corresponding device for the heat treatment of the sections is to be provided for. It has been determined that such an extension of the device according to the invention tends to increase the complexity and hence the cost of the device, associated with a decrease in robustness at the same time. Additionally, the sections are partly exposed to different heat treatments (e.g. in the microwave, the steamer or the steam cooker) regarding the primary anti-body to be used and preferably different buffer solutions are also used in practice. In the following, examples for preferential varying treatment of sections in using different primary-antibodies are listed:

HSP 90: 10 min. Proteinase K: 1 drop. PK (DAKO Cytomation, Code No.S 2019) in 2 ml Dilution medium (DAKO Cytomation, Code No. S 2032) with RT.

CyclinA: 45 minutes cooking in steam cooker (MultiGourmet, Firma Braun) with citric acid (2 g Citric Acid Monohydrate/1.0 I A.d., pH6).

Jaw 1/Ki-67: 20/15 minutes pressure cooker with citric acid (2 g Citric Acid Monohydrate/1.0 l A.d., pH 6).

Therefore devices that according to invention are not designed for pretreatment of tissue or cell sections are preferred.

The reagents for immunohistochemical and molecular biological proof reactions are commercially available and are chosen with regard to the desired examination method. Currently, approximately 250 well-established antibodies are available for immunohistochemical analyses which can especially be used for the immunohistochemical standard-staining method, the so called Avidin-Biotin-Complex-(ABC-) method.

The dosing head or the dosing head holder is preferably equipped with a cooling device e.g. formed by Peltier-elements. This allows for stability of reagents in the reservoirs of the dosing head over a long period. Alternatively, it is proposed to provide a cooling station similar to the refill station into which the dosing head can be moved via the positioning device. This is preferably as the mass of the dosing head to be moved can be distinctly reduced compared to the embodiment with dosing head comprising a cooling station, thus the position device can be dimensioned more cost-effective. Accordingly, the dosing head is automatically transferred into the cooling station, subsequent to the staining passage and the reagents left in the reservoirs of the dosing head are cooled in order to prolong the period of usage. Generally, the primary and secondary antibodies and the tertiary reagent required e.g. for the ABC-method are relatively stable in an adequate buffer solution and if cooled, they can be stored over a period from 1 to 4 weeks up to 6 or 12 months.

In a preferred embodiment of the device, which does not contain a dosing head comprising a cooling device or a cooling station into which the dosing head can be moved, can be equipped with a draining or storing function. Subsequently to a staining procedure and the removal of the slides from the device, reservoirs are preferably inserted into the slide reception devices or other adequate receptions (trays) into which the reagents which are still in the reservoirs of the dosing head are completely drained by means of the metering valve. Preferably these reservoirs are marked with identification elements, for instance 1D- or 2D-codes which can be read by the reading device at the dosing head. Hence, it is assured that the dosing head dispenses the reagents' residues in the appropriate storage containers. Subsequently, these reservoirs can be removed and stored in a remote cooling device.

With the ABC- method, the primary-antibody binds the corresponding tissue-antigen in the first step. Appropriate primary-antibodies are e.g.

CyclinA (Novocastra Laboratories Ltd., Klon 6E6): 1:50

Hsp90 α/β (N-17, sc-1055, Santa Cruz Biotechnology, Inc.): 1:150

Jaw1 (E-19, sc-11688, Santa Cruz Biotechnology, Inc): 1:150

Ki-67(Cione Mib-1,DAKO Cytomation, M7240, Denmark): 1:100

whereby the dilution of the primary-antibody onto the mentioned concentration is conducted with e.g. “ChemMate” Antibody Diluent (e.g. DAKO Cytomation S 2022). The thus diluted primary-antibody can then be metered into the corresponding reaction zones of the slides. The reaction time of the primary-antibody is e.g. 20 to 30 min.

This is followed by one rinsing step or a plurality of rinsing steps, preferably followed by a slight drying step which must not desiccate the cell or tissue sample in the reaction zones.

In the following step a biotinylated secondary-antibody is bound to the primary-antibody. As secondary-antibody reagents the following may be exemplarily listed:
For mouse-primary-antibody (CyclinA):
Goat-anti-Mouse IgG Plus (Biocare Medical, Cat #GM601MMplus), ready for use
For goat-primary-antibody (HSP90 & Jaw1):
Mouse-anti-Goat IgG (Jackson Immuno Research Laboratories Inc., 205-065-108), to be diluted with ChemMate Antibody Diluent (DAKO Cytomation S 2022) onto 1:50

The reaction time of the secondary-antibody is e.g. set to 10 to 20 min.

Several rinsing and preferably subsequent drying steps can be followed by a blocking step, whereby the endogen Peroxidase-activity is blocked with a Peroxidazed blocking agent (Biocare Medical, Cat #PX968MM) according to the principle of a H₂O₂surplus with missing electron donor. The reaction time of the blocking reaction is set to e.g. 3×5 min.

After several rinsing and preferably subsequent drying steps, the preformed Avidin-Biotin-Enzyme-Complexes are applied as tertiary reagent (as enzyme used: horseradish peroxidase (HRP) or Alkaline Phosphatase (AP)) and its highly affine adsorption to the biotin-molecule of the secondary-antibody.

The reaction time of the tertiary reagent is set to e.g.10 to 20 min.

The several rinsing and preferably subsequent drying steps are followed by the incubation with a chromogene substrate solution, whereby e.g. 3.3′-Diaminobenzidin-Tetrahydrochlorid (DAB) (DAB Chromogene (Biocare Medical, Cat #DB851-60) or Neufuchsin can be used as chromogene by forming a visible brown or red reaction product. The chromogene solution can preferably contain a buffer medium (e.g. DAB Substrate Buffer (Biocare Medical, Ref DS854MM; Chromogene to buffer medium in relation 1:20).

The reaction time of the chromogene is set to e.g. 5 to 10 min.

After several rinsing and preferably subsequent drying steps the reaction zones of the slides can be treated with Haematoxylin (reaction time e.g. 1 min.). The cell and tissue samples in the reaction zones of the slides can then be dehydrated (ascending alcoholic sequence: 70%-70%-96%-100% Ethanol for 1 min respectively) and be treated with Xylol. The slides can then be sealed with a cover for storage.

The exemplary described reagents and further possible reagents in a rprepared state are filled into the reservoirs of the dosing head. The reagents in the reservoirs are preferably arranged in that a simultaneously dosing of the reagents is possible depending on the respective analysis step. The reagents can be arranged e.g. in blocks of 6, i.e. in rows of 6 antibodies in a defined sequence, thus resulting in useful panels.

The following table exemplifies arrangements of 6 reagents (antibodies) respectively, in panels which are compatible to staining algorithms and can be used advantageously for pathological diagnostics of each of the mentioned lesions/tumours.

| Leucemia and | | klz. B-cell | Plas- | neuroen- | | Mama | | |
|---|---|---|---|---|---|---|---|---|
| precursors | | lymphom | macytom | docrinical | | Ca | | |
| I | II | I | II | tumours | KM Ca | I | II | III |
| CD3 | CD10 | CD5 | CD20 | KL1 | KL1 | CK7 | CK14 | CK5/6 |
| | | | | | (A1/A3) | | | |
| CD34 | CD14 | CD20 | CD38 | NSE | ER/PR | p6 | CK5/6 | CK14 |
| CD79 | CD56 | CD23 | CD56 | TTF1 | CK7 + 8 | ER | p53 | Calretinin |
| CD117 | KP1 | Kappa | Kappa | MIB1 | CEAp | PR | HmFg | Synapto |
| MPO | Glyco | Lambda | Lambda | Synapto | PSA | Her2neu | Cyclin d1 | |
| TdT | Lysozym | Cyclin D1 | | Ceromo | S100 | E- | gCDFP | |
| | | | | | | Cadherin | | |

The stated abbreviations are standard denotations for commercially available antibodies. If applicable, the antibodies can be diluted in the conventional manner and be metered in defined sequence in the reservoirs of the dosing head. The resulting algorithmic panel can be connected to a separately manageable dosing head segment which is inserted separately from other dosing head segments into the dosing head holder to be completed as a dosing head. To avoid confusion the dosing head segments that are comprised in panels are preferably marked or mechanically and/or electrically encoded.

Further staining algorithmic panels can be seen in table 1 given below.

According to the invention the device is especially adapted to conduct the method of dosing the reagents onto a slide or into the slots of the slides, respectively.

For this purpose the slides are inserted into the device. Preferably, the slides are arranged on one tray or a plurality of trays outside the device. The trays are then inserted into the device. The slides are fixed onto the trays with mechanical fastening elements, preferably with a mechanical fastening system with female and male fastening elements. The slides preferably comprise labelling fields whereto an identification medium such as e.g. etiquettes with 1D- or 2D-Codes, RFID-Chips or similar mediums can be attached. Preferably, the alignment of the slides is always the same in that the operator can rapidly verify that all labelling fields show in the same direction.

Whether the slides with the sections are facing up and their labelling fields are all directed the same, is the only check which can be performed by the operator at this stage. The sequence of the slides on the inventive device is arbitrary as all slides are read-out by an integrated scanner first of all. Hence, the device can immediately check if a slide has been inserted, which currently has no suiting panel with reagents (in the dosing head). False processing of this slide can hence be excluded. Furthermore, an optimized travelling path can be calculated for the dosing head if necessary.

Furthermore, the method comprises the filling of the reservoirs of the dosing head with adequate reagents. The dosing head is then attached to the positioning system or inserted into the dosing head holder which is attached to the positioning system. Preferably, the dosing head or the dosing head holder have a cooling device which comprises e.g. Peltier-elements. If the device contains a refill station for reagents at which the dosing head can be refilled after being emptied, said station is also filled with reagents. The reservoirs of the dosing head as well as the reservoirs of the refill station are marked with identification elements in order to permit an automatic control of the analysis, i.e. the dosing of the reagents into the slots or the reaction zones and the refilling of the dosing head can be monitored by software. Additionally, it is preferably ensured that the reagent volume in the dosing head or in the refill station correlates with the totality of reaction zones.

Subsequent to these preparations the analysis-run is started. The dosing head with the reading device for the identification means moves along the slides in order to read the information on the identification means and to determine the position of the slides and of their slots/reaction zones. Afterwards the dosing head starts to dose reagents into the slots/reaction zones. The volume of the reagent to be dosed is smaller than the volume of the slots/reaction zone. The dosing head follows the sequence of reaction steps with the intermediate rinsing steps etc. predefined by the respective analysis-method as e.g. the above described ABC-method.

Additionally, the method preferably comprises a step at which the slides or the slots/reaction zones located on the slide respectively are covered with a protective sheet to protect them for the subsequent analysis steps as e.g. microscopic analyses.

### BRIEF DESCRIPTION OF THE FIGURES

A specific embodiment of the device and the dosing head according to the invention is described on the basis of the enclosed figures,

FIG. 1 top view of a slide to be used according to the invention,

FIG. 2 a cross-sectional view of a slide along the dotted line A-A drawn in FIG. 1,

FIG. 3 a perspective full view of the device according to the invention with closed cover,

FIG. 4 a perspective full view of the device according to FIG. 3 with open cover,

FIG. 5 a perspective full view of the dosing head of the device according to FIGS. 3 and 4,

FIG. 6 a perspective detailed view of the dosing head according to FIG. 5,

FIG. 7 another perspective full view of the dosing head, and

FIG. 8 a view of the dosing head from underneath.

### DETAILED DESCRIPTION OF THE FIGURES

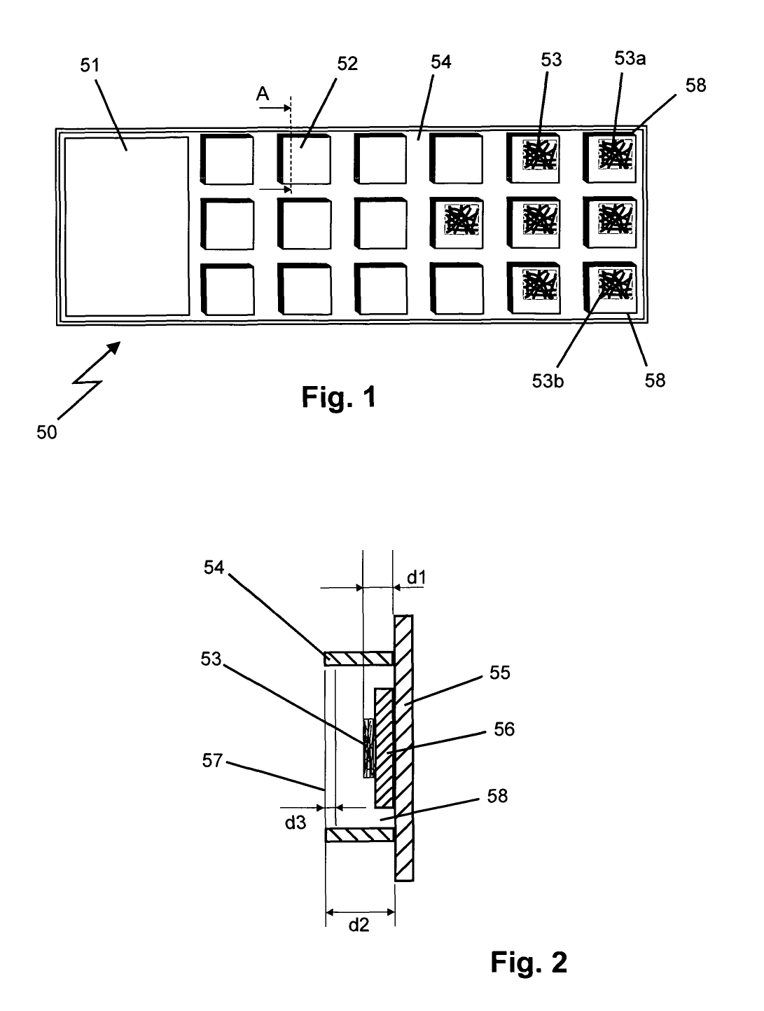

The slide 50 shown in FIG. 1 has slots 52 with a quadratic cross-sectional area which are arranged in a regular 3×6-Matrix. The slots 52 are equidistantly arranged in rows of 3 and columns of 6. The distance between the rows is larger than between the slots 52 in a row itself. The slots 52 each contain a tissue or cell sample 53, 53a, 53b. The slots form the reaction zone 58. The slide 50 shows a labelling field 51 on which a 1D- or 2D-etiquette can be attached which identifies the slide 50.

FIG. 2 shows a cross-sectional view of a reaction zone 58 along the dotted line A-A drawn in FIG. 1. FIG. 1 shows a quadratic slot 52 without a tissue or cell sample 53 whereas FIG. 2 shows a tissue or cell sample 53 for clarification. The tissue or cell sample 53 is placed on a carrier foil 56 composed of a polycarbonate foil and is attached onto the basis plate 55 of the slide 50. The slide 50 is for example coated with sprayed-on frame consisting of a hydrophobic material (reference 54) as e.g. Teflon foil into which the slots 52 or reaction zones 58 are embedded. The frame or the foil 54 respectively have a thickness of d2 which is larger as the sum d1 of the thicknesses of the cell or tissue samples 53 and the carrier foil 56, so that the tissue or cell samples positioned in the slots 52 can be impinged with reagents without the risk of a contamination of adjacent slots 52. The relation of the dimensions d2/d1 is preferably at least 1.2, more preferably at least 1.3 and more preferably at least 1.4. The volume of the slots exceeds the sum of the volumes of the tissue and cell samples 53 and the carrier foil segments by at least 1.5 and preferably by at least 2.

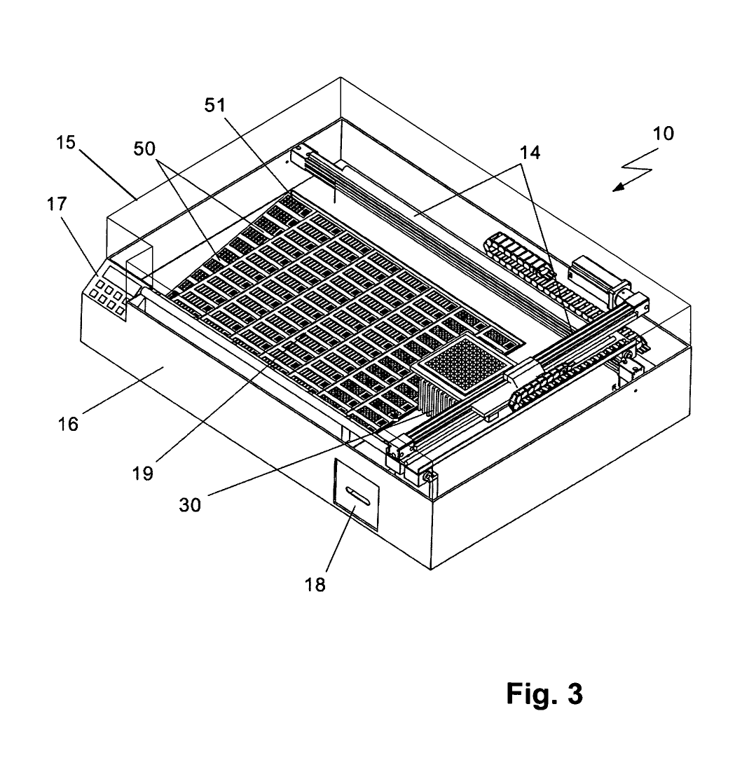

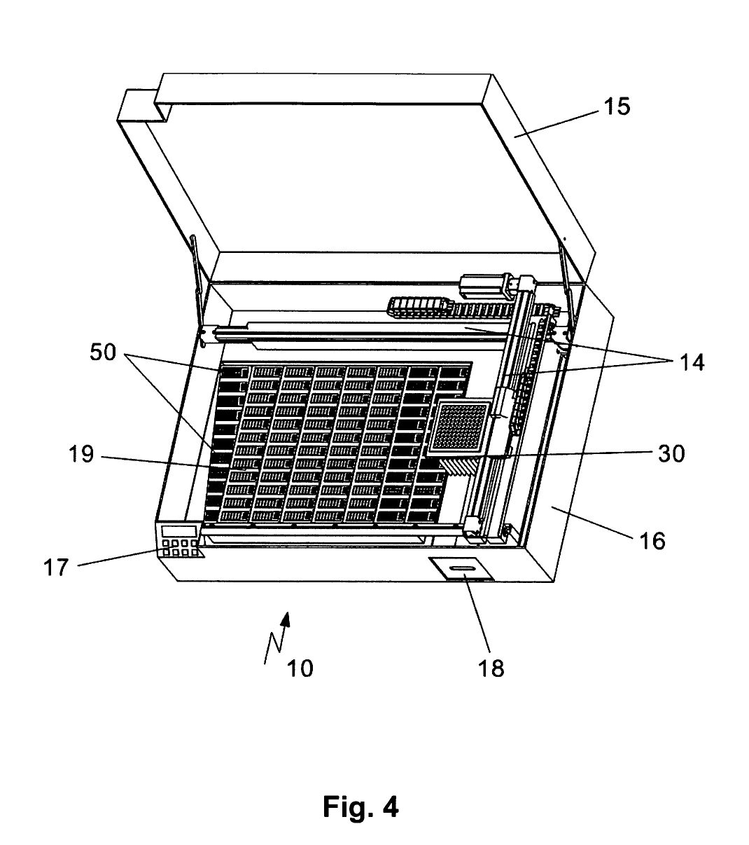

If desired, the slide 50 can be covered with a protective sheet 57 (with a thickness of d3) prior to the start of the reactions to ensure that the cell or tissue samples 53 do not desiccate. The protective sheet 57 will be removed before conducting the proof reactions. FIG. 3 and FIG. 4 show a perspective full view of an exemplary embodiment of a device 10 according to the invention. The device 10 is loaded with a multitude of slides 50 which are arranged in a regular matrix (12 rows×8 columns). The 12 slides 50 of a column are attached to a strip-shaped tray 51. One end of the tray 51 is can be lowered and submerges at the lowered state in the rinsing tank which is positioned under the slides 50 arranged at one level. The rinsing tank is located in the housing of the device 10 and cannot to be seen in FIGS. 3 and 4.

The installations for the in- and outlet of the rinsing fluid are located behind the lid 18. The device 10 can be sealed with a transparent cover sheet, whereby a damp location is generated in the reaction room 19 above the level of the slides 50. This ensures a continuous humidification of the cell of tissue samples 53 in the reaction zones of the slides 50. The cover sheet 15 is shown in FIG. 3 in a closed stated and in FIG. 4 in an opened state.

Additionally, the device 10 contains a dosing head 30 which is attached to an arm of the Cartesian X/Y-positioning system 14 and can hence be positioned above the slides 50. The arms of the Cartesian system 14 are mounted at two sides respectively which results in a robust positioning system. The operation of the device is done via the control-panel 17.

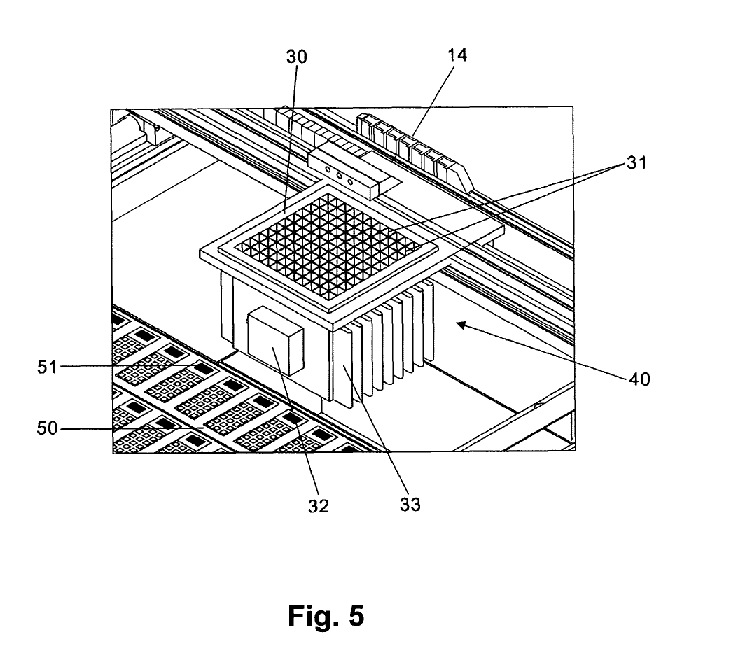

FIG. 5 shows a perspective view of the dosing head 30 which is inserted into a dosing head holder 40 which is attached to an arm of the Cartesian X/Y positioning system 14. The dosing head contains a multitude of reservoirs 31 which have a quadratic cross-sectional area and are arranged in a quadratic matrix (11×11). The holder 40 of the dosing head 30 is equipped with Peltier-cooling elements 33 and has a barcode reader 32 on the side facing the reaction room 19 by means of which the barcode etiquettes located in the labelling fields 51 of the slides 50 can be read.

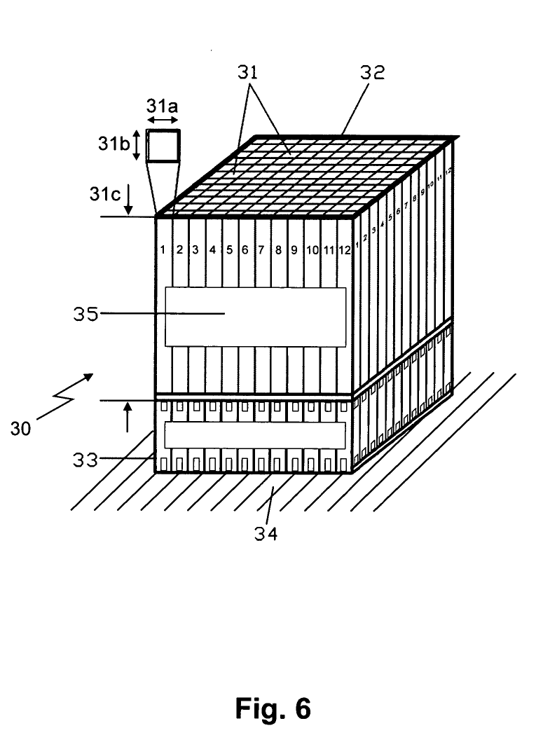

FIG. 6 shows the principal construction of the dosing head 30 with quadratic reservoirs 31 which are arranged in a quadratic 12×12 matrix. The dosing head 30 comprises 144 reservoirs 31 in total, each having a quadratic cross-section and the same volume. The individual reservoirs 31 at both ends of the dosing head are numbered 1 through 12. The detailed view shows the quadratic cross-sectional area of a reservoir 31 with the side lengths 31a, 31b. A metering valve 33 is attached at the opposite end of each reservoir 31. The metering valves 33 are aggregated in a dosing block. The metering valves 33 have an outlet device for the reagents inside the individual reservoirs. A block of flow sensors 34 is connected to the metering valves 33 to control the dosing. The height of the reservoirs 31 between the upper open ends, which have a gastight cover 32 and the metering valves 33 located in the dosing block 30 is identified as 31c.

The reservoirs 31 have a gastight cover at the end which faces the metering valves 33. The installation for the gas conduit is not shown in the schematic view of FIG. 6. The reservoirs 31 can be impinged e.g. with compressed air or an inert gas by means of the gastight cover 32. Below the metering valve block the block 34 with the flow sensors is located in order to verify the flow or rather the dosing.

The dosing block 30 has a labelling field 35 to facilitate the exchange of dosing heads 30 within the dosing head holder 40. In an exemplary dimensioning the reservoir in FIG. 6 shows side lengths 31a, 31b of 9 mm each and a height 31c of ca. 45 mm. This results in a volume of approximately 3.6 ml per reservoir 31 and a total volume of the dosing head 30 of about 525 ml.

As shown in FIG. 6 the dosing head 30 is constructed “sandwich”-like with exchangeable plates or blocks. It consists of a gastight cover 32, of reservoirs 31 combined into a block unit, of a metering valve 34 block and/or of a block of flow sensors. This permits a high maintainability and modularity for potential extensions.

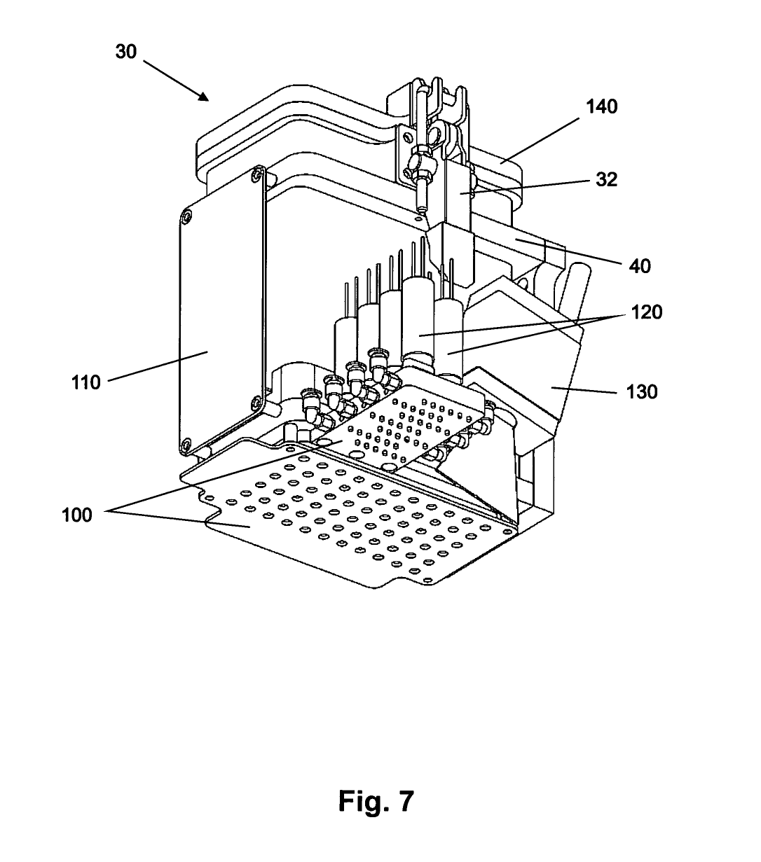

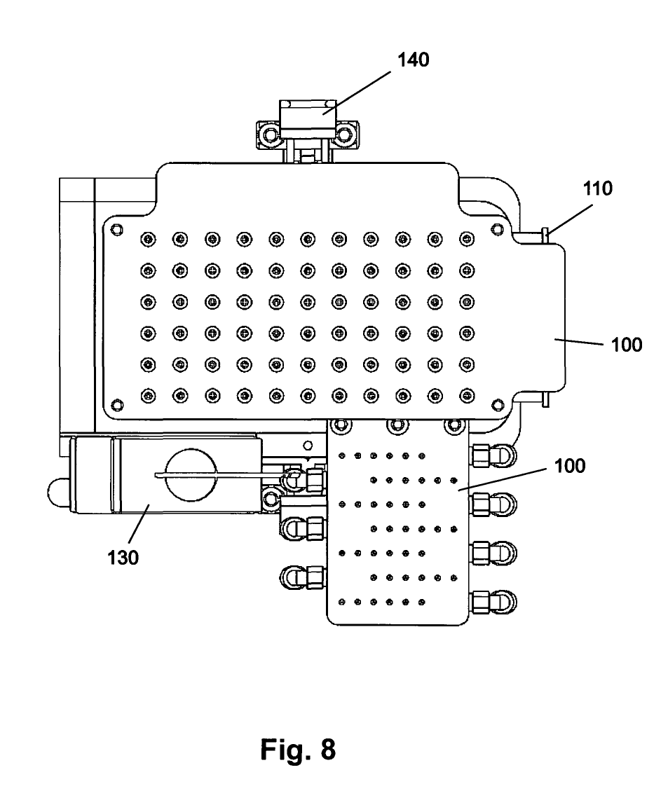

The dosing head 30 shown in FIG. 7 and FIG. 8 is inserted into a dosing head holder 40. The dosing head 30 contains besides the dosing control 100 as well an electronic system 110 to activate the valves of the dosing head 30 and the additionally required valves 120 for the liquids which are used in large quantities per slide 50. Furthermore, the electronic system 110 includes a pressure sensor to verify the system pressure (not shown). The scanner 130 shown in FIG. 7 and FIG. 8 is used to identify and evaluate the 2D-codes located on the slides 50. The cover 32 in its closed position is secured against unintentional opening by a closing mechanism 140 which is located on both sides in the upper area of the cover.

TABLE 1
staining-logarithmic panels for pathologic diagnostics of the indicated carcinoma

| Precursor | | klz. B-cell | | | | | |
|---|---|---|---|---|---|---|---|
| and leucemia | | lymphom | Plasmocytom | klz. Bly + Pz | k. T-cell | | |
| I | II | I | II | I + II | lymphom | Hodgkin | NLPHD |
| CD3 | CD10 | CD5 | CD20 | Cd5 | CD3 | CD15 | CD3 |
| CD34 | CD14 | CD20 | CD38 | CD10 | CD4 | CD20 | CD23 |
| CD79 | D56 | Cd23 | D56 | CD20 | CD8 | CD30 | D57 |
| CD117 | KP1 | Kappa | Kappa | CD23 | D56 | LMP1 | EMA |
| MPO | Glyco | Lambda | Lambda | CD38 | CD57 | PAX5 | TIA |
| TDT | Lysozym | Cyclin D1 | | CD56 | TIA | OPD | 4 |
| | | | | CD79 | | | |
| (CD1a) | | | | Kappa | | (CD2) | (CD2) |
| (CD5) | | | | Lambda | | (TIA) | (TIA) |
| (CD7) | | | | Cyclin D! | | (EMA) | (EMA) |
| (CD19) | | | | MIB | | | |
| (CD22) | | | | TdT | | | |
| (CD33) | | | | | | | |
| (CD99) | | | | (CD25) | | | |
| (CD123) | | | | (TRAP) | | | |
| (IgM) | | | | (IgD) | | | |
| (PAX) | | | | (DBA44) | | | |
| Hg-B | | Hg-T | | Histocytic | unknow | CA | |
| I | II | I | II | tumour | tumour | I | II |
| CD5 | CD23 | CD3 | CD2 | CD1a | CD38 | KL1 + AE | CDX2 |
| | | | | | | 1/3 | |
| CD10 | CD30 | CD4 | CD8 | CD23 | CD45 | CK5 + 6 | CEA |
| CD20 | CD79 | CD20 | CD56 | S100 | CD56 | CK10 + 13 | CA19.9 |
| MIB | OCT2 | CD30 | TIA | KP1 | CD138 | TTF1 | CA12.5 |
| bd2 | IgM | MIB | Granzym | Lysozym | VIM | CK7 + 8 | Calretin |
| | | Alk | Lysozym | | S100β | ER + | Synapto |
| | | | | | | PR | |
| | | | | | KL1 | | |
| (EBER( | (EBER) | (alpha/ | (alpha/ | (EBER) | | (CD14) | |
| | | beta) | beta) | | | | |
| | | | | | | (Her2neu) | (Her2neu) |

| neuroen- | | | | | | | |
|---|---|---|---|---|---|---|---|
| docrine | | MamaCa | | gamete | Sacrome | | neurogene |
| tumours | KMCa | I | II | tumour | I | II | tumours |
| KL1 | KL1 (P1/A3) | CK7 | CK14 | CD30 | CD34 | CD31 | GF AP |
| NSE | ER | p63 | CK5/6 | AFP | CD99 | EMA | MIB1 |
| TTF1 | PR | ER | p53 | PLAP | CD117 | Demin | S100 |
| MIB1 | CEAp | PR | HmFg | EMA | S100 | smActin | MAP |
| Synapto | PSA | Her2neu | Cyclin d1 | KL1 | VIM | Myogenin | EMA |
| Ceromo | S100 | E-Cadherin | gCDFP | Inhibin | KL1 (AE 1/3) | Calretinin | PR |
| | CK7 + 8 | CK5/6 | Synapto | CD117 | | | |
| | | CK14 | | | | | |
| | | Calretinin | | | | | |
| | | (Her2neu) | (Her2neu) | (NSE) | | | |

1. Automatic device to meter reagents onto a multitude of slides comprising
holding devices for a multitude of slides each containing a plurality of separated slots which are adapted to absorb cell or tissue samples,
a dosing head unit with a plurality of reservoirs which are adapted to absorb liquid reagents and which have a metering valve at the end/side facing the slots to dose and apply the reagents into the slots, and
a positioning device to position the reservoirs of the dosing head unit above the respective slots of the slides.

2. Device according to claim 1, whereby the quotient of the reservoirs' average volume and the slots' average volume is at least 50.

3. Device according to claim 1, whereby the average volume of the slots is between 5 and 500 pl.

4. Device according to claim 1, whereby the slots have an average depths of less than 2 mm and a cross-sectional area of less than 100 mm².

5. Device according to claim 1, whereby the average volume of the reservoirs is at least 1.5 ml and preferably at least 2.5 ml.

6. Device according to claim 1, whereby the slots are framed by bars and separated from each other.

7. Device according to claim 1, whereby the slots form a regular array.

8. Device according to claim 7, whereby the slots are arranged in stripes or in a regular matrix.

9. Device according to claim 1, whereby a cell or tissue sample is arranged in each slot.

10. Device according to claim 1, whereby the slides are placed mainly at the same level.

11. Device according to claim 1, whereby the slides are detachably mounted onto one tray or a plurality of trays.

12. Device according to claim 11, whereby the slides are removably attached to the tray by a locking system with male and female closure elements.

13. Device according to claim 1, whereby the trays can be detachably inserted into the device.

14. Device according to claim 1, whereby the cross-sectional area of adjacent reservoirs is chosen in adaptation to the cross-sectional area of the slots such that two slots of two adjacent reservoirs can be impinged simultaneously with reagents.

15. Device according to claim 1, whereby the reservoirs are dimensioned in such way that at least three, preferably six adjacent slots can simultaneously be impinged with proof-reagents out of the reservoirs.

16. Device according to claim 14, whereby the reservoirs are filled with reagents of a staining-logarithmic panel.

17. Device according to claim 1, hereby the reservoirs have a cross-sectional area of preferably less than 1 cm².

18. Device according to claim 1, whereby the outlet of the metering valve is arranged above the openings of a perforated plate.

19. Device according to claim 1, whereby the outlet of the metering valve is equipped with a dosing needle.

20. Device according to claim 1, whereby the dosing head unit includes flow sensors/transducers to measure the volume of the metered reagents.

21. Device according to claim 1, whereby the individual metering valve and/or the dosing needle comprises a device to monitor the bubbles.

22. Device according to claim 1, whereby the end of the reservoirs opposite of the metering valve can be gas-tightly closed.

23. Device according to claim 1, whereby the device has a holder in which one or a plurality of dosing heads can be inserted.

24. Device according to claim 1, whereby the dosing head unit is detachably connected to the dosing head holder.

25. Device according to claim 1, whereby the dosing head unit and/or the dosing head holder are equipped with cooling elements.

26. Device according to claim 1, whereby the device preferably has a cooling station in which the dosing head can be moved via the positioning device to cool the reagents contained in the reservoirs.

27. Device according to claim 1, whereby the device preferably includes a automatic draining-station in which the dosing head can be moved via the positioning device to completely drain the dosing head and to store the drained reagents.

28. Device according to claim 1, whereby the dosing head and/or the dosing head-holder comprises a reading device for identification elements located on the slides.

29. Device according to claim 1, whereby the device comprises a rinsing tank located below the slides to absorb the rinsing fluid.

30. Device according to claim 1, whereby a slide individually or together with other slides is transferable from a first position adapted to dose the reagents into the slots to a second position located in the rinsing tank.

31. Device according to claim 30, whereby the tray carrying the slides individually or together with other trays is transferable from a first position adapted to execute proof reactions to a second position located in the rinsing tank.

32. Device according to claim 1, whereby the device includes a cover located above the slides.

33. Device according to claim 1, whereby the positioning system is a Cartesian Y/Y-System.

34. Device according to claim 1, whereby the device contains a dosing head holder in which one or several dosing heads can be inserted.

35. Device according to claim 1, whereby the device preferably includes a automatic filling station in which the dosing head with the partly or completely drained reservoirs can be moved by the positioning device to refill the reservoirs with the respective reagents.

36. Dosing head to be used in an automatic device to meter reagents onto a multitude of slides comprising a multitude of reservoirs which are adapted to absorb liquid reagents and which at one end/side contain a metering valve to dose and apply the reagents onto the slides and at the other end opposite to the metering valve contain a gastight cover, whereby the average volume of the reservoirs is at least 1.5 ml and preferably at least 2.5 ml.

37. Dosing head according to claim 36, whereby the average volume of the reservoirs is preferably at least 5 or 7 ml.

38. Dosing head according to claim 36, whereby the reservoirs are filled with reagents of a staining-logarithmic panel.

39. Dosing head according to claim 36, whereby the reservoirs show a cross-sectional area of preferably less than 1 cm².

40. Dosing head according to claim 36, whereby the outlet of the metering valves is arranged above the openings of the perforated plate.

41. Dosing head according to claim 36, whereby the outlet of the metering valve is equipped with a dosing needle.

42. Dosing head according to claim 36, whereby the dosing head contains a dosing control to measure the volume of the drained reagents. The dosing control is configured primarily by optical flow sensors or by a capacitive flow measurement system.

43. Dosing head according to claim 36, whereby the individual metering valve and/or the dosing needle comprise a device to control the bubbles.

44. Dosing head according to claim 36, whereby the dosing head comprises a holding device to absorb liquid and especially proof reagents such as pipes conducting secondary and/or tertiary reagents, whereby holding device preferably comprises a metering valve and/or a dosing control device.

45. Dosing head according to claim 36, whereby one or a plurality dosing heads are configured in that they can be detachably inserted into the dosing head-holder of the automatic device to dose the reagents onto a multitude of slides.

46. Dosing head according to claim 36, whereby the dosing head is equipped with cooling elements.

47. Dosing head according to claim 36, whereby the dosing head comprises a reading device for the identification elements located on the slides.

48. Method to dose reagents onto a multitude of slides by means of an automatic device according to claim 1, whereby
the slides are inserted into the device,
the reservoirs are filled with reagents,
the dosing head is positioned over the respective slots of the slides by means of the positioning device, and
the reagent is dosed into the slot at which the volume of the reagent is smaller than the volume of the slot.

49. Method according to claim 48, whereby the slides are marked with identification elements and the dosing head or the dosing head-holder of the device contain a reading device to read the identification elements. In doing so the dosing head moves the slides before the reagents are drained in order to read the information on the identification elements and to adjust the position of the slides and of the slots provided for by the slides.
